(19) United States
(12) Patent Application Publication
(10) Pub. No.: US 2020/0088715 A1
(43) Pub. Date: Mar. 19, 2020

(54) ARCHITECTURE FOR COAGULATION ANALYSIS WITH TISSUE FACTOR

(71) Applicant: Hewlett-Packard Development Company, L.P., Spring, TX (US)

(72) Inventors: Uranbileg DAALKHAIJAV, Corvallis, OR (US); Rachel M. WHITE, Corvallis, OR (US); Tod WOODFORD, Corvallis, OR (US); Chantelle DOMINGUE, Corvallis, OR (US)

(73) Assignee: Hewlett-Packard Development Company, L.P., Spring, TX (US)

(21) Appl. No.: 16/494,473

(22) PCT Filed: Apr. 20, 2017
(86) PCT No.: PCT/US17/28586

§ 371 Date: Sep. 16, 2019

(51) Int. Cl. G01N 33/49; G01N 33/86

(52) U.S. Cl. CPC G01N 33/4905; G01N 33/86

(57) ABSTRACT

A microfluidic blood coagulation testing die includes a substrate, a slot in the substrate permitting entry of a sample of blood, a chamber in the substrate for collection of cells in the sample, and a pinch point that permits passage of sample from slot to chamber. The die includes a sensor in or near the at least one pinch point to test sample passing through the at least one pinch point. Activator, such as freeze-dried coagulation-initializing tissue factor, coats at least a portion of the interior surface of the slot, pinch point, and chamber.

### BACKGROUND

The blood coagulation cascade is a complex biological process involving a sequence of chemical reactions that finally result in a clot. Blood coagulation measurement may be used, for example, by patients on oral anti-coagulant treatment (e.g., warfarin) for conditions such as atrial fibrillation, deep vein thrombosis, and congenital heart defects. Clotting time may be quantified, for example, as prothrombin time (PT) or an International Normalized Ratio (INR). For some such patients, routine testing is often necessary to monitor for proper coagulation capability and changes in therapeutic range as may result from a variety of factors, including diet and metabolism.

A convenient coagulation test device that could be used at a primary care physician's office or in-home can provide an attractive alternative to hospital laboratory testing for patients requiring constant PT/INR monitoring to ensure that they stay within a moderate anticoagulant intensity as provided by an appropriate treatment dosage.

### BRIEF DESCRIPTION OF THE DRAWINGS

FIG. 1 system block diagram depicting an example of a microfluidic coagulation testing system.

FIG. 2 is a plan view of an example architecture of a microfluidic clotting testing device.

FIG. 3 is a cross-sectional diagram of an example architecture of a microfluidic clotting testing device.

FIGS. 4-6 are plan views of different example architectures of a microfluidic clotting testing device.

FIGS. 7-8 are plan views of different examples of a pinch point of a microfluidic clotting testing device.

FIG. 9 is a data plot illustrating an example test measurement made by an example microfluidic clotting testing device.

FIG. 10 shows two plots illustrating analysis to determine clotting time.

FIGS. 11 and 12 are flowcharts showing example clotting time analysis methods.

Throughout the drawings, identical reference numbers designate similar, but not necessarily identical, elements.

### DETAILED DESCRIPTION

This disclosure provides a cost-effective, handheld microfluidic device, capable of quickly and reliably measuring PT/INR value from extremely small volume of blood (e.g., less than five microliters), as may be obtained, for example, by finger prick. The devices and methods described herein permit for very small amounts of coagulation-initializing tissue factor to be used in each single-use testing device (e.g., less than two hundred nanoliters of tissue factor). The devices and methods described herein further permit for more rapid and more accurate measurement of blood clotting time.

As used herein, the term “fluid” is meant to be understood broadly as any substance, such as, for example, a liquid or gas, that is capable of flowing and that changes its shape at a steady rate when acted upon by a force tending to change its shape.

Also, as used herein, the term “microfluidic” is meant to be understood to refer to devices and/or systems having channels sufficiently small in size (e.g., less than a few millimeters, including down to the nanometer range) such that surface tension, energy dissipation, and fluidic resistance factors start to dominate the system. Additionally, use of the term “microfluidic” is used to indicate scales at which the Reynolds number becomes very low, and side-by-side fluids in a straight channel flow laminarly rather than turbulently. In some examples, a microfluidic channel is less than one millimeter in width as measured at a cross-section normal to the net direction of flow through the microfluidic channel. In other examples, the width of a microfluidic channel is less than five hundred micrometers, such as less than two hundred micrometers or less than one hundred micrometers.

Unless specified to the contrary or otherwise made plain by context, references to “channels” or “pumps” should be understood to refer to microchannels and micropumps, respectively.

Further, as used herein, the term “a number of” or similar language is meant to be understood as including any positive integer.

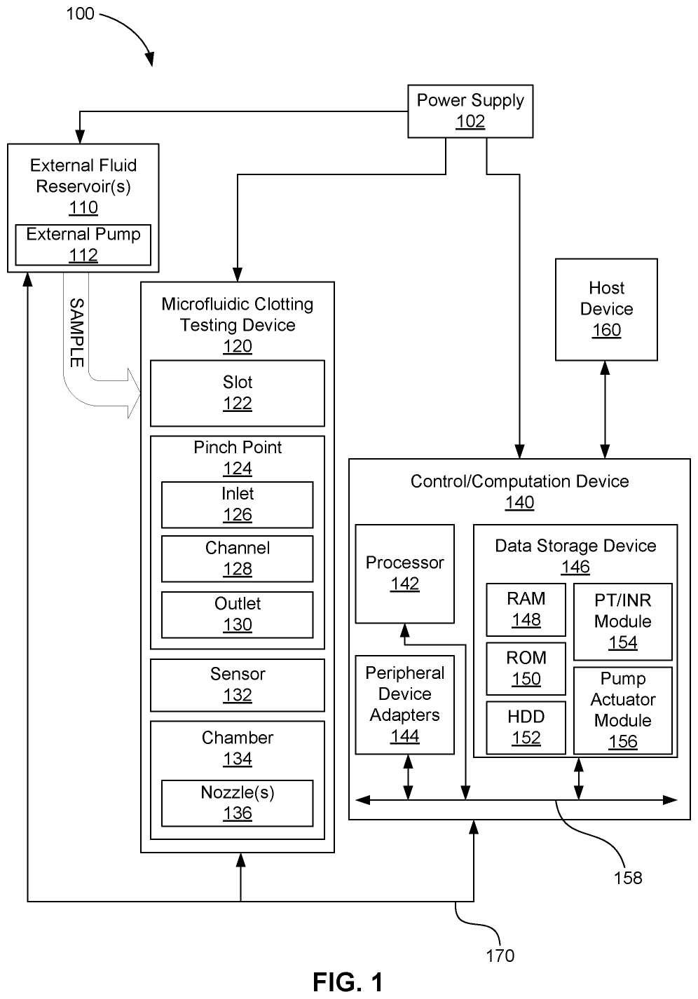

FIG. 1 is a system block diagram depicting an example of a microfluidic coagulation testing system 100. The microfluidic coagulation testing system 100 can accept a sample of fluid (e.g., blood) into microfluidic clotting testing device 120 to determine the clotting rate as represented by such metrics as PT and/or INR. Device 120 may be fabricated, for example, using wafer fabrication manufacturing processes and techniques. The sample may be accepted directly from a subject, as by fingerprick, for example, or may be accepted from an earlier draw which may, for example, be stored in one or more external fluid reservoirs and pumped into microfluidic clotting testing device 120 via one or more external pumps 112.

Regardless of how sample is introduced into device 120, sample (e.g., whole blood from a finger stick) can enter slot 122 and can flow through at least one microfluidic “pinch point” 124 into a chamber, sometimes called a “foyer,” 134. Each pinch point can include one or more portions, including a slot-side sample entry portion, or inlet, 126, a middle portion, or channel, 128, which can be of substantially consistent width and height, and a chamber-side exit portion, or outlet, 130.

After sample has passed through pinch point 124, all or part of it may collect in chamber 134. Chamber 134 can include one or more nozzles 136 to assist in removal of air and/or fluid from chamber, thereby to promote flow of sample through pinch point 124 and particularly its channel 128. The one or more nozzles 136 can include or have associated therewith one or more micropumps (not shown) to aid removal of air and/or fluid, which micropumps can be any type of micropump, capillary or inertial. In some examples, device 120 can omit chamber 134 and instead pump sample fluid that has passed through pinch point 124 into, for example, a separate waste receptacle, or simple eject it from device 120.

At least one sensor 132 in or near pinch point 124 can test sample passing through pinch point 124. For example, sensor 132 (or several such sensors working on combination) can measure or detect flow of sample through pinch point 124. By “near,” it is meant that no part of sensor 132 is more distant than one hundred micrometers from the inlet or outlet of the pinch point, e.g., no more distant than fifty micrometers, e.g., no more distant than ten micrometers. As one example, sensor 132 can measure or detect electrical resistance across all or a portion of pinch point 124 to produce electrical resistance data that can serve as a basis for determining some metric related to flow. As another example, sensor 132 can measure or detect optical transmittance to produce optical transmittance data that can serve as a basis for determining some metric related to flow. As yet another example, sensor 132 can measure or detect pressure to produce pressure data that can serve as a basis for determining some metric related to flow. As still another example, sensor 132 can be a magnetic sensor that can measure or detect magnetic flux to produce magnetic flux data that can serve as a basis for determining some metric related to flow. In some examples, the sensor 132 is operated at a sample rate on the order of milliseconds. In some examples, the sensor 132 is operated at a sample rate on the order of microseconds. The portion of device 120 in which sensor 132 is operative is herein referred to as the “sense zone.” In some examples, the sense zone may include substantially all of pinch point 124, but in some examples the sense zone may include only a portion of pinch point 124 and/or may include portions of slot 122 and/or chamber 134.

Whatever type of data or signals may be derived from sensor 132, such data or signals can be sent or transmitted, wired or wirelessly, to control/computation device 140, which can include a processor 142 and storage 146. Data or signals can be transmitted, for example, over signal lines 170. Signal lines 170 may also be used to transmit signals or instructions from control/computation device 140 to device 120 and/or reservoir 110.

The data storage device 146 may store data and/or instructions such as executable program code that is executed by the processor 142 or other processing device. The data storage device 146 may specifically store a number of applications that the processor 142 can execute to implement at least the functionality described herein. The data storage device 146 may comprise various types of memory modules, including volatile and nonvolatile memory. For example, the data storage device 146 can include one or more of random-access memory (RAM) 148, read-only memory (ROM) 150, flash solid state drive (SSD) (not shown), and hard disk drive (HDD) memory 152. Many other types of memory may also be utilized, and the present disclosure contemplates the use of many varying type(s) of memory in the data storage device 146 as may suit a particular application of the principles described herein. In certain examples, different types of memory in the data storage device 142 may be used for different data storage needs. For example, in certain examples the processor 142 may boot from ROM 150, maintain nonvolatile storage in the HDD memory 152, and execute program code stored in RAM 148.

In this manner, the control/computation device 140 includes a programmable device that includes machine-readable or machine-usable instructions stored in the data storage device 146, and executable on the processor 142 to make determinations of sample coagulation time and/or related parameters, and/or to control microfluidic clotting testing device 120, for example, to control any pumps that may be in or associated with its nozzles 136. For example, storage 146 may store one or more modules, such as a PT/INR module 154 to make determinations of PT and/or INR values from signals or data received from sensor 132, and/or a pump actuator module 156 to implement sequence and timing instructions for selectively activating and deactivating the pumps as may be in or associated with nozzles 136.

In some examples, the control device 140 may receive instructions, signals and/or data from a host device 160, such as a computer, and temporarily store the instructions, signals and/or data in the data storage device 146. The instructions, signals and/or data from the host 160 can represent, for example, executable instructions and parameters for use alone or in conjunction with other executable instructions in other modules stored in the data storage device 146 of the control/computation device 140 to control fluid flow, analysis output, and other related functions within the microfluidic coagulation testing system 100 and its microfluidic clotting testing device 120.

For one example, the instructions, signals and/or data executable by processor 142 of the control/computation device 140 may timely enable and disable pumping by pumps to promote flow of sample through pinch point 124. For another example, the instructions, signals and/or data executable by processor 142 of the control/computation device 140 may read and store signals and/or data from the sensor(s) 132 and analyze or process such signals and/or data to arrive at values indicative of clotting time, such as PT and/or INR values.

Hardware components of control/computation device 140 may be interconnected through the use of a number of busses and/or network connections. In some examples, the processor 142, data storage device 146, and peripheral device adapters 144 may be communicatively coupled via bus 158.

The processor 142 may comprise the hardware architecture to retrieve executable code from the data storage device 146 and execute the executable code. The processor 142 can include a number of processor cores, an application specific integrated circuit (ASIC), field programmable gate array (FPGA) or other hardware structure to perform the functions disclosed herein. The executable code may, when executed by the processor 142, cause the processor 142 to implement at least the functionality of the external pump 112 (if any), nozzle(s) 136 and/or associated pumps (if any), and microfluidic clotting testing device 120, such as disclosed herein. In the course of executing code, the processor 142 may receive input from and provide output to a number of the remaining hardware components, directly or indirectly.

The processor 142 may also interface with a number of sensors, such as sensor 132, or may otherwise measure, calculate, or estimate the flow rate of fluid flowing through the punch point 124. For example, the processor 142 may calculate or estimate the flow rate of sample flowing through the pinch point 124 based on known factors including the electrical resistance of discrete features of the sample, e.g., the electrical resistance of individual blood cells, or without any a priori knowledge, simply by looking at a signal from the sensor over a period of time. As examples, control/computation device 140 can determine that the flow rate through the pinch point 124 has fallen below a predetermined threshold level or otherwise has changed with reference to an earlier measured flow rate. Alternatively or additionally, control/computation device 140 can determine the start and stop of clotting time by observing and statistically testing signal variance, as described herein.

The microfluidic coagulation testing system 100 may also comprise a number of power supplies 102 to provide power to the external fluid reservoir(s) 110 and external pump(s) 112 (if present), the microfluidic clotting testing device 120 nozzles 136 and their associated pumps (if present), and the control/computation device 140, along with other electrical components that may be part of the microfluidic coagulation testing system 100.

In some examples, the microfluidic clotting testing device 120 and its elements may be implemented as a chip-based device that can include slot 122, pinch point 124, sensor 132, and chamber 134 with outlet nozzles 136, or combinations thereof. The structures and components of the microfluidic clotting testing device 120 may be fabricated using a number of integrated circuit microfabrication techniques such as electroforming, laser ablation, anisotropic etching, sputtering, dry and wet etching, photolithography, casting, molding, stamping, machining, spin coating, laminating, among others, or combinations thereof.

In some examples of the devices and systems described herein, the microfluidic clotting testing device 120 and/or associated components can be fabricated in a one-time use, disposable component. Such a disposable component can be removable, modular, and replaceable.

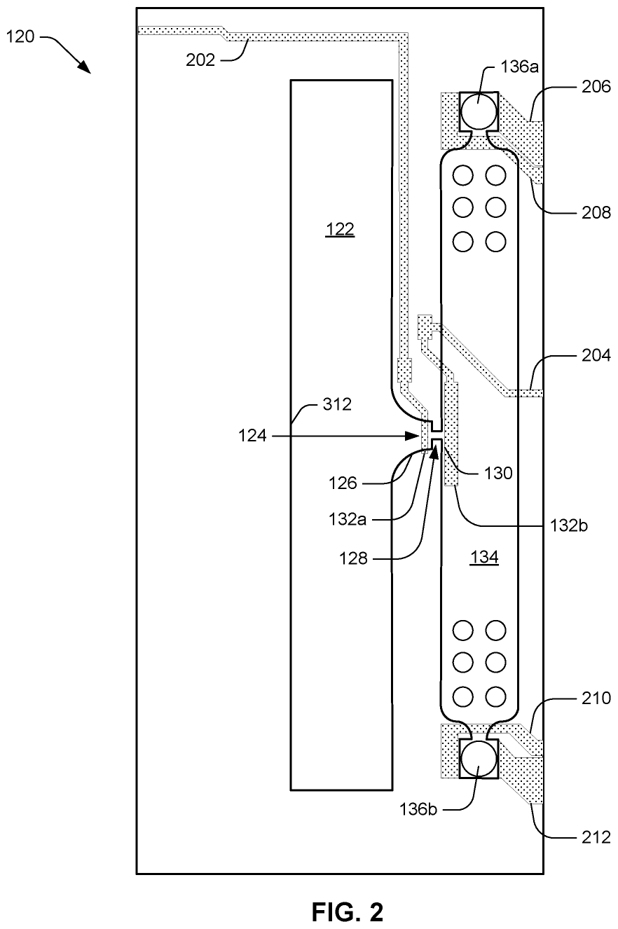

FIG. 2 illustrates an example architecture of the microfluidic clotting testing device 120. A sample of fluid (e.g., blood) can enter via slot 122, flowing, for example, in a direction perpendicular to the plane of the drawing. Sample is induced to enter and flow through pinch point 124 in the direction indicated by the arrow associated with reference numeral 124 by either or a combination of positive pressure (slot-side) or negative pressure (via pull from chamber 134, e.g., via the draw provided by air-liquid interfaces of nozzles 136a, 136b). Pinch point 124 includes a squeezed channel 128 where sensor 132 for measuring red blood cell flow can be roughly located. The size of channel 128 helps determine the sensitivity to cell flow and affects the likelihood of clogging.

Following passage through pinch point 124, sample can collect in chamber 134, and in some instances, liquid portion of sample (e.g., blood plasma) can be drawn out through nozzles 136a, 136b. Chamber 134 can be sized to allow cells to fill without backing up into pinch point 124. For example, the chamber can be large enough to allow continuous filling of red blood cells from undiluted whole blood sample for at least two minutes. Chamber 134 may be sized, for example, to collect thousands of red blood cells during a test. Thus, chamber 134 promotes red blood cell packing for the duration of the coagulation test. Unhindered packing of the chamber until sufficient measurement data to compute prothrombin time has been collected from sensor 132 can be essential for gathering a useful measurement data set from sensor 132.

Nozzles 136a, 136b can include holes in the microfluidic chamber 134, the size and location of which act as a driving force for wetting and the speed of cell flow. Nozzles can be, for example, of the type used as thermal ink-jet pumps in ink-jet printers. In many instances, sample may consist of discrete features in a carrier fluid (e.g., red blood cells in blood plasma). The evaporation of carrier fluid (e.g., plasma) at an air-liquid interface (e.g., meniscus) can drive the movement of the discrete features (e.g., cells) toward the air-liquid interface where evaporation is occurring, i.e., toward the nozzles 136. In such cases, the nozzles provide passive promotion of flow. In some examples, however, nozzles can provide active flow by providing each nozzle with one or more pumps to eject fluid. For example, nozzles can include firing resistor to eject fluid out of nozzles, which can hasten the testing process.

In addition to promoting migration of discrete sample features through pinch point 124 during a test, nozzles 136 can also promote evaporation and clumping of activator (e.g., tissue factor) during the voidage coating and freeze-drying process that can be part of the fabrication process of device 120. Nozzles 136a, 136b can be located on either side of the chamber 134 to promote discrete feature (e.g., red blood cell) flow and packing. Each nozzle 136a, 136b can be less than sixty micrometers in diameter and can be located away from the sense zone so that red blood cell packing velocity is not high enough to promote lysing, and red blood cell drying signal does not reach the sensor 132 in the pinch point 124. In some examples, no nozzle 136 is located within one hundred micrometers of the pinch point outlet 130. In some examples, no nozzle 136 is located within thirty micrometers of the pinch point outlet 130.

An activator can be used to initialize coagulation at a certain point in the coagulation cascade. It may be that an activator is added to sample prior to introduction to device. However, such an added step may be inconvenient. Thus, in some examples of device 120, all or a portion of its voidage may be internally coated, as a part of the fabrication process, with an activator, e.g., a freeze-dried coagulation initializing tissue factor, to trigger a transformative process in the sample under test, e.g., the clotting cascade in blood. As an example, 25% Dade Innovin tissue factor may be introduced into slot 122 in liquid form and freeze-dried in the device 120 to preserve protein activity for subsequent reaction with sample, and to initiate fibrin formation upon wetting by sample. Dade Innovin is one example activator, but any coagulation activator suitable to the test may be used. When freeze-dried, the tissue factor can form a fluffy and spindly structure (not shown) inside the voidage that can wet instantly and evenly when exposed to sample.

Architectural features of device 120 can address issues that arise from the above-described internal coating of device 120 with activator. The activator's coating of walls can result in a higher concentration of activator within the pinch point 124 and around ports and nozzles 136. Resultantly, sample may experience a faster rate of fibrin formation at locations of higher local concentration of tissue factor, e.g., in the pinch point 124 and around ports and nozzles 136. Clogging of the pinch point 124 can occur when the width W of the pinch point is too small (e.g., less than ten micrometers). It is therefore important that the pinch point 124 is appropriately shaped and sized in examples that are to be coated with tissue factor. Such examples may also be constructed to have a reduced number of ports and nozzles 136, e.g., no more than two. Moreover, any posts in the architecture should be in slot 122 only.

The respective surface areas of the features of device 120 can be sized to minimize the necessary coating with activator while still providing adequate surface area for tissue factor coating and sufficient volume for sample flow. For example, slot 122 can be made to be no greater than 1,200,000 square micrometers in surface area, pinch point 124 can be made to be no greater than 300 square micrometers in surface area, and chamber 134 can be made to be no greater than 40,000 square micrometers in surface area. For example, slot 122 can be made to be between 800,000 and 1,200,000 square micrometers in surface area, pinch point 124 can be made to be between 160 and 240 square micrometers in surface area, and chamber 134 can be made to be between 34,000 and 38,000 square micrometers in surface area. For example, slot 122 can be made to be 1,000,000 square micrometers in surface area, pinch point 124 can be made to be 200 square micrometers in surface area, and chamber 134 can be made to be 36,000 square micrometers in surface area.

In the architecture illustrated in FIG. 2, the aforementioned sensor comprises two electrodes 132a, 132b arranged near the inlet 126 and outlet 130 of pinch point 124, i.e., on either side of microchannel 128. Electrodes 132a, 132b are thereby arranged to measure an electric field between inlet 126 and outlet 130, which electric field is concentrated within pinch point 124. In some examples, the electrode 132a closer to the slot 122 serves as a ground electrode. Electrical leads 202, 204 can respectively connect electrodes 132a, 132b to other circuitry (not shown) for, e.g., amplification, filtering, and eventual delivery to control/computation device 140. Electrical leads 206, 208 can provide electrical power to control nozzle 136a and/or to power a pump associated with nozzle 136a, while electrical leads 210, 212 can provide similar functionality for nozzle 136b and/or an associated pump. In the example shown in FIG. 2, inlet 126 is illustrated as having a funnel shape.

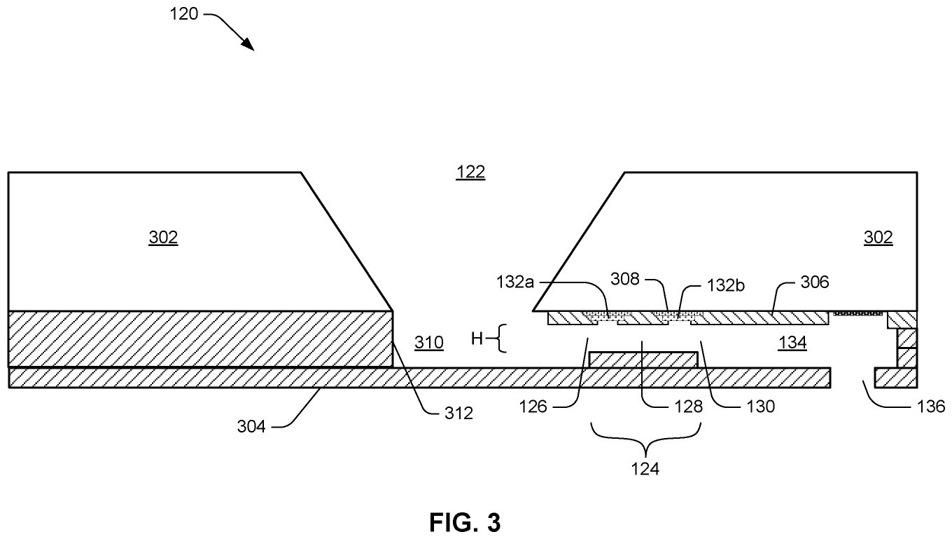

FIG. 3 is a cross-sectional diagram of an example architecture of the microfluidic clotting testing device 120. As shown in FIG. 3, slot 122 can taper into main reservoir or passage 310 where sample can flow through pinch point 124 into chamber 134. Similar to the arrangement shown in FIG. 2, electrodes 132a, 132b can be arranged near inlet 126 and outlet 130 of pinch point 124, i.e., on either side of channel 128. Layer 302 can be, for example, bulk silicon, through which slot can be etched. Additional layers 304 can be, for example, thin-film deposited using polymer, e.g., SU-8 polymer, which can be made transparent so as to make the preparation of pinch point 124 with activator visually inspectable and its functioning during a test visually monitorable. SU-8 is an epoxy-based negative photoresist that is highly biocompatible. Polydimethylsiloxane (PDMS) is another example polymer that may be used to deposit additional layers 304. Insulating layers 306, 308 can insulate electrodes 132a, 132b and their associated traces from other layers of device 120. One port or nozzle 136 is illustrated in FIG. 3. Because FIG. 3 shows a cross-section, the particular shape or features of inlet 126 and outlet 130, if any, may not be noted in FIG. 3. FIG. 3 does, however, note height H of pinch point channel 128.

In both FIGS. 2 and 3 it may be noted that pinch point 124 and chamber 134 appear on only one side of slot 122, i.e., only on the left side as illustrated in these drawings. In some examples, another pinch point and chamber can be placed on the opposite side of slot 122, more or less in mirror image of pinch point 124 and chamber 134 as illustrated in FIGS. 2 and 3. However, the arrangement shown, with no mirror-image pinch point and chamber, can improve sample pressure and thus flow of sample through pinch point 124. Stated another way, the presence of sample flow-blocking wall 312 on the opposite side of slot 122 from pinch point 124 can force sample to channel into chamber 134 on the open side of slot 122.

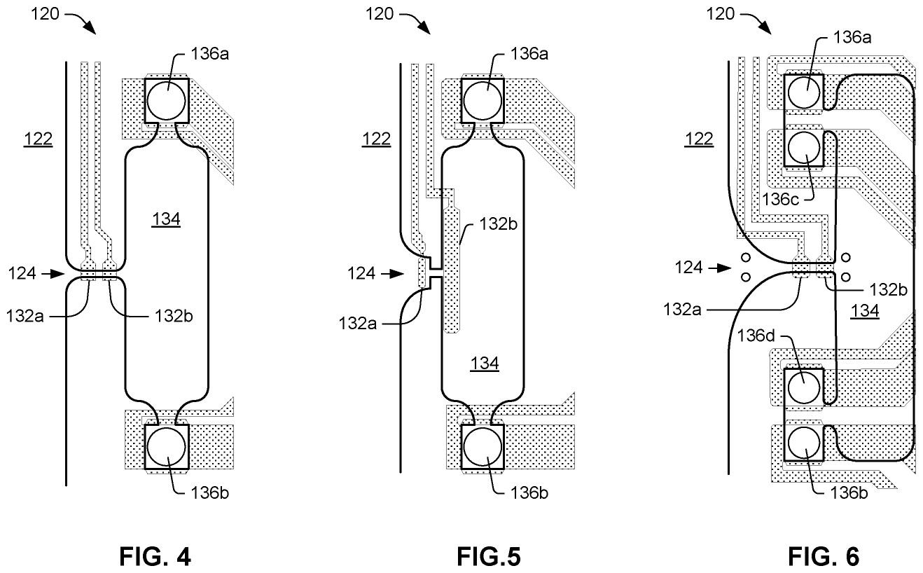

FIGS. 4-6 illustrate, by way of three different examples, variations that may be present in the architecture of microfluidic clotting testing device 120. FIG. 4 shows a pinch point 124 with funnel-shaped inlet and outlet. In FIG. 4, electrodes 132a, 132b are arranged within the pinch point 124, on opposite sides of its channel 128 (label omitted to preserve clarity). FIG. 5 shows a pinch point arrangement similar to that illustrated in FIG. 2, with electrode 132a situated in the inlet of pinch point 124 and electrode 132b situated outside of the pinch point 124, near its outlet, in the chamber 134. Although pinch point inlet is funnel-shaped in FIG. 5, a right angle leads into the pinch point's channel, while there are no such right angles in the architectures of FIGS. 4 and 6. The funnel-shaped inlet to pinch point 124 in FIG. 6 has a much larger mouth and edge radius than the funnel-shaped outlet from pinch point 124 in FIG. 6. As in FIG. 4, in FIG. 6, electrodes 132a, 132b are arranged within the pinch point 124, on opposite sides of its channel 128 (label omitted to preserve clarity). Additionally, the chamber 134 in the architecture of FIG. 6 features four nozzles 136a-d, instead of two nozzles, as shown in the other examples.

The arrangement and size of electrodes 132a, 132b can determine the sensitivity of sensor 132 to discrete sample features, e.g., individual red blood cells, as opposed to detecting bulk sample flow. Examples having smaller electrodes 132a, 132b arranged inside pinch point 124 can be more sensitive to passage of discrete features through pinch point 124, whereas examples having larger electrodes 132a, 132b arranged further apart, e.g., outside pinch point, will be less sensitive to transits of individual discrete features but will instead measure bulk flow.

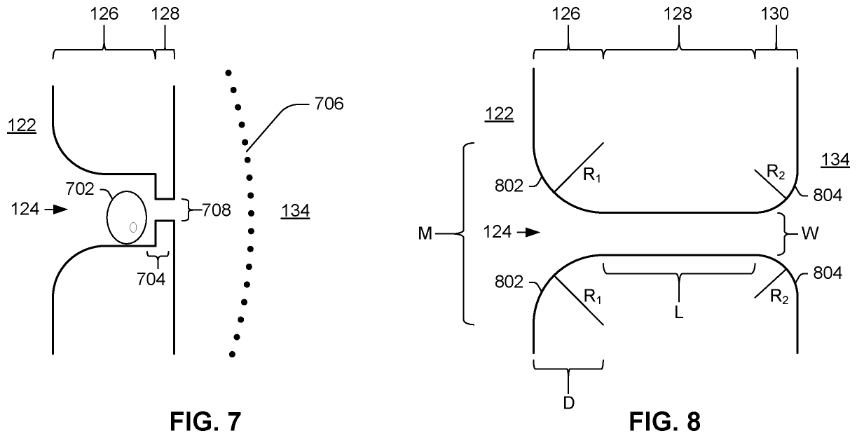

FIGS. 7 and 8 illustrate various shape features of pinch point 124. Sensors, nozzles, and other features are omitted for the purposes of illustration. Like the architectures shown in FIGS. 2 and 5, FIG. 7 illustrates a pinch point 124 with a funnel-shaped inlet 126 but with right angles 704 between the inlet 126 and the channel 128 of pinch point 124. Right angles 704 can promote trapped bubble formation when blood sample wets activator (e.g., freeze-dried tissue factor). As sample passes from slot 122 through pinch point 124 into chamber 134, as illustrated by wetting front 706, an air bubble 702 can form and become trapped by right angle 704, potentially impeding flow of sample through pinch point 124 and providing inaccurate readings of flow and clotting time. Due to the small pinch point width 708 and the air bubble 702 in the pinch point, the speed of die wetting as sample plasma coagulates is drastically reduced.

By contrast, the pinch point 124 in FIG. 8 has no right angles between inlet 126 and channel 128. In FIG. 8, pinch point 124 has funnel shaped inlet 126 and outlet 130. For example, funnel-shaped inlet can decrease from a mouth width M to channel width W over a distance D. The inlet edges can be of constant slope, can have piecewise changing slope, or, as illustrated, the inlet edges 802 can have a gradually changing slope. For example, and as illustrated in FIG. 8, the edges 802 can have the shape of a quadrant of a circle of radius R₁. Similarly, the outlet edges can have same or (as illustrated) different radius R₂. Channel 128 can have substantially consistent width W and height (perpendicular to the plane of the drawing) over the length L of channel 128. In the illustrated example, mouth width M is equal to 2R₁+W, and inlet length 126, D, is equal to R₁. Similarly, in example illustrated in FIG. 8, the outlet length 130 is R₂. The funnel-shaped pinch point 124 with rounded corners 802, 804 allows sample plasma to wet the die quickly and smoothly. The speed and evenness of the initial wetting prevents air bubbles from forming and allows red blood cells to fill the chambers, with reduced risk of clogging the pinch point 124. In some examples, pinch point 124 is hourglass-shaped in at least one view, e.g., in a plan view. By “hourglass-shaped,” it is meant that pinch point 124 has both a funnel-shaped narrowing inlet 126 and a funnel-shaped widening outlet 130.

The dimensions of pinch point 124 and its inlet 126, channel 128, and outlet 130 can be tailored to the particular application of device 120. Moreover, as noted above, the size of the pinch point 124 can be designed to prevent clogging or cell plugs from forming within pinch point 124. Additionally, as can be seen in FIGS. 7 and 8, the pinch point inlet 126 can be funnel-shaped to allow cells to flow into the chamber smoothly without sharp obstructive angles (e.g., right angles 704) which may promote bubble formation and cell clumping.

The dimensions of pinch point 124 can be sized and shaped to permit for good pre-coagulation sample flow, even when the pinch point is internally coated with activator, but without being so large that sensor 132 measures bulk sample flow as opposed to flow of discrete sample features, e.g., individual red blood cells. In some examples, therefore, pinch point channel width W is about the width of one, or a few, human red blood cells. In some examples, channel width W is no greater than fifteen micrometers. For example, channel width W can be between ten and fifteen micrometers. As another example, channel width can be between six and eight micrometers. In some examples, pinch point channel height H, as illustrated in FIG. 3, is of substantially identical size as width W. In some examples, channel height H is no greater than fifteen micrometers. For example, channel height H can be between ten and fifteen micrometers. As another example, channel width can be between six and eight micrometers.

Fibrin can cause blood to transition from liquid to gel and ultimately to solid, thus to form a clot. Because it is desirable, during a test of blood coagulation, that clotting occur within pinch point 124 in order to achieve a clear cut-off of sample flow as visible in measurement data collected from sensor 132, channel length L can be made to be no longer than necessary to have a large enough sense zone and for fibrin to form within pinch point 124. In some examples, channel length L is no greater than fifteen micrometers. For example, channel length L can be between five and fifteen micrometers.

In some examples, inlet 126 narrows from a mouth width M of twenty micrometers to a narrower width of ten micrometers within a length of ten micrometers. In other examples, inlet 126 narrows from a mouth width M of thirty micrometers to a channel width W of ten micrometers within an inlet length D of ten micrometers.

Although the illustrated examples show only a single pinch point and chamber, in some examples device 120 can include multiple pinch points leading to a chamber, or multiple pinch points leading to multiple chambers, emanating from the slot. Multiple sensors in the multiple pinch points can yield multiple readings, the results of which can be reported separately or averaged or otherwise combined to yield a single output.

The systems and devices described above thus include, as one example, a microfluidic coagulation testing die that includes a slot in a substrate permitting entry of a sample, a sample collection chamber in the substrate, and at least one pinch point connecting the slot and the chamber, permitting passage of sample from slot to chamber. The pinch point can have a slot-side inlet, a channel, having a length, of substantially consistent width and height, the width being between ten and fifteen micrometers and the height being between ten and twenty micrometers, and a chamber-side outlet. In some examples, the pinch point have no angled corners in the inlet or channel in a plan view. The pinch point can have a sensor in or near the pinch point. Coagulation-initializing activator can coat at least a portion of an interior surface of any or all of the slot, the pinch point, and the chamber.

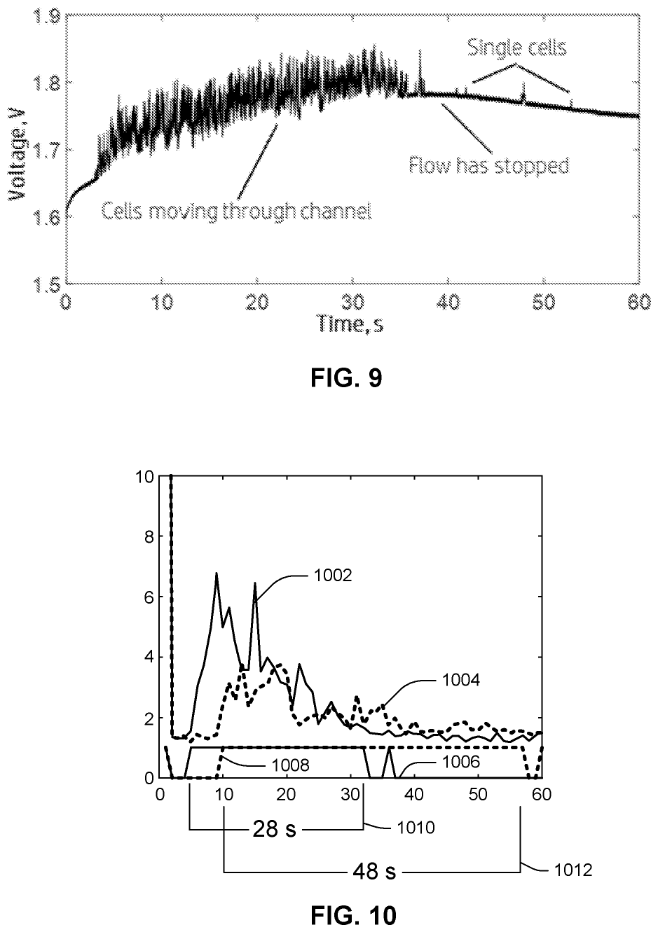

FIG. 9 illustrates a plot of an example set of measurements of the electrode-type sensor illustrated in the preceding drawings FIGS. 2-6 represented as a potential difference between electrodes 132a, 132b, measured in volts, over time, in seconds. A lower potential difference can represent a lower resistance between the two electrodes, while a higher potential difference can be indicative of discrete sample features (e.g., blood cells) passing through pinch point 124 and thus between electrodes 132a, 132b. For example, as red blood cells move through pinch point 124, they can create a peak in the electric signal generated by sensor 132. Each peak in the data set illustrated in FIG. 9, then, is indicative of individual cells or small groups of individual cells flowing between electrodes 132a, 132b of sensor 132. As may be observed, determining coagulation time involves, in essence, an observation of the time it may take for peaks to stop appearing in the data set generated by sensor 132 over the course of the test. Data processing may be used to ascertain a clotting time and various metrics from the collected data set.

Referring still to FIG. 9, as a cell passes through pinch point 124 and over sensor 132, the voltage measured across the electrodes 132a, 132b increases because red blood cells are resistive compared to the surrounding blood plasma. When cells are flowing over the sensor, a series of peaks appear in the collected measurement data, indicating smooth red blood cell flow through pinch point 124. This is the condition observed between about the three-second mark and the thirty-seven-second mark in the plot of FIG. 9 (“cells moving through channel”). As coagulation occurs, the blood plasma transitions from a liquid to a gel and traps red blood cells. This is the condition observed at about the 40-second mark (“flow has stopped”). Although a few individual cells may sporadically make it through the channel, noted as isolated peaks between the forty-second mark and the fifty-five-second mark (“single cells”), these individual cell transits are nevertheless few and infrequent enough to conclude that the clotting process was completed.

For a given sample of blood and a collected data set of the type illustrated in FIG. 9, coagulation time metrics, such as PT and/or INR values, can be obtained by the analysis method now described. The data collection can be obtained using a microfluidic device, e.g., a chip, containing an architecture sensitive to red blood cell flow, e.g., using the system 100 and/or device 120 described above. Sample may be introduced to the microfluidic device 120 to begin the test. For each test of sample, a first analysis phase of the method may produce a raw PT value and a second phase may derive an INR value. In the manner described above, following the introduction of sample into device 120, sensor signals indicative of discrete sample features (e.g., passage of individual blood cells) can be collected by sensor 132. For example, where sensor 132 comprises a pair of electrodes 132a, 132b, voltage signals can be collected from device 120 for a set period of time. The set period of time should be greater than the expected coagulation time for the sample. In the case of human blood, a sufficient test time is usually about two minutes, even if such blood is anticoagulated.

Raw sensor signals collected by sensor 132 can be passed on to additional conditioning and processing circuitry, which can include filtering, amplification, and operation circuitry. In some examples this circuitry may be implemented as part of device 120 during fabrication of device 120. As an example, a low-pass filter can be applied to the raw sensor signals to obtain filtered signals. The filtered signals can then be subtracted from the raw sensor signals to obtain unbiased signals.

Then, for a series of predetermined small time intervals (e.g., every one second), the signal variance can be computed from the unbiased signals, to yield a “piece-wise” variance. The current variance (at time t) can be compared with the previous variance (at time t−1 interval) to conclude if the current variance of the signal is significantly increased, using an appropriate statistical test, such as chi-squared hypothesis testing. The result of this statistical test can be a binary decision.

If the variance is significantly increased as established by the chosen test, the current time t may be marked as the beginning of the coagulation process. Otherwise, the variance computation and comparison may be repeated until a significant variance increase arises, marking coagulation onset.

Once coagulation onset is established, the current variance (at time t) may be computed and compared with the previous variance (at time t−1 interval) to conclude whether the current variance of the signal is significantly decreased or increased, again using an appropriate statistical test, such as chi-squared hypothesis testing. Again, the result of this statistical test can be a binary decision.

If the variance is significantly decreased or increased, the post-coagulation-onset variance computation and comparison may continue. If, however, variance is determined to be stable following a period of steadily declining variance, the current time t may be marked as the end of the coagulation process, whereupon the first phase may be terminated and the time difference between the end of the coagulation process and its onset may be determined to be the raw PT value, and may in some instances be recorded and/or reported as such, e.g., via output to host device 160 from control/computation device 140.

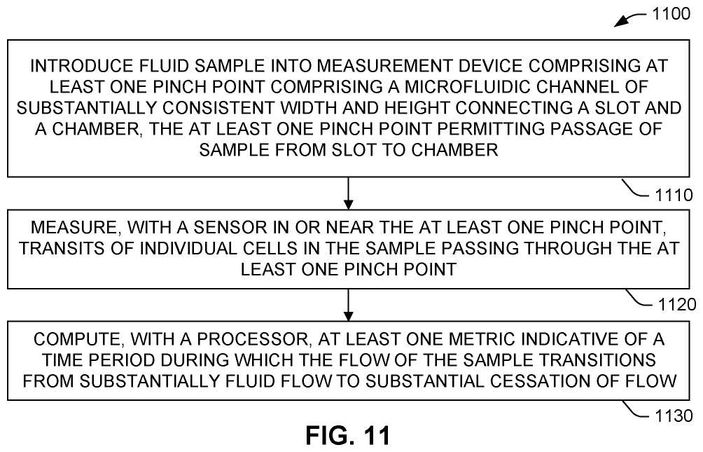

FIG. 10 illustrates the first phase of the analysis process (i.e., the computation of a raw PT value from collected data) for two different test trials, one using regular whole blood (plots 1002, 1006) and one using blood from a patient on anticoagulant therapy (plots 1004, 1008). Plots 1002, 1004 represent, for the two different trials, average variances computed from sensor signals in the manner described above. Such computation can be performed, for example, by control/computation device 140 using, for example, PT/INR module 154. For both trials, a large drop in resistance occurs near time zero indicative of wetting of the sense zone with carrier fluid (e.g., plasma). This is followed by an increase signal variance as discrete features (e.g., red blood cells) begin to traverse pinch point 124 and thus enter the sense zone.

Plots 1006, 1008 represent the binary decision outputs of the chosen statistical test on the variance, as discussed previously, for corresponding signals 1002, 1004, respectively. Accordingly, these are plotted exclusively as either zero or one. As can be seen in FIG. 10, unanticoagulated trial variance 1006 rises at about the four to five second mark, indicative of the onset of heavy red blood cell flow at the beginning of the test, and falls at about the thirty-two to thirty-three second mark, indicative of clotting. The difference 1010 between the marked times, in this case twenty-eight seconds, represents the determined raw PT value. By contrast, anticoagulated trial variance 1008 rises at about the nine to ten second mark and falls at about the fifty-seven to fifty-eight second mark, resulting in a difference 1012 of forty-eight seconds, which is expectedly longer than the clotting time 1010 in the unanticoagulated trial.

To summarize, the onset of the coagulation process coincides with the beginning of fluid through the sense zone. For device architectures using dual-electrode type sensors, this wetting of the sense zone results in a large drop in voltage across the electrodes, and thus a very high variance in the signal. An unchanging variance following a decline in variance after the initial variance increase marks the conclusion of coagulation. If no decrease in variance is detected throughout the test for a set (long enough) period of time, it means there is no coagulation at all. Absent this unusual scenario, the pattern of variance will generally resemble the plots 1002, 1004 illustrated in FIG. 10, i.e., (1) a period of low variance, (2) followed by a short burst of high variance, (3) followed by a steady decline in variance, and finally (4) a long period of steady unchanging variance.

The raw PT value derived by the above method can be empirically correlated to a standardized PT value as may be produced by a different method and/or test apparatus using, for example, a linear function. Resultantly, the raw PT value can be converted using such a function for storage or output. Such conversion function can be stored, for example, in storage 146 and such conversion can be performed, for example, by control/computation device 140.

In the second phase of the analysis of collected sensor data, a non-linear empirical function may be applied to this raw PT value to obtain the standard INR value. For example, the INR can be determined from the obtained PT by evaluating the following i^{th} order polynomial conversion equation:

INR=a₀+a₁×t+a₂×t²a₃×t³+ . . . +aᵢ×tⁱ

where t is the raw PT value obtained from the above-described method, and the function parameters a₀ through aᵢ can be calculated using data from several blood tests done based on various blood types with distinct INR values, measured by a standard benchmark device, e.g., an FDA-approved device. Plotting the INR data for various blood types against the device-specific PT results in a curve (e.g., a 2^{nd}-order polynomial) that can be used to compute the function parameters a₀, a₁, a₂, etc., using a least squares curve-fitting technique. Once the function parameters have been obtained, arbitrary PT values computed using the above method can be plugged in to the above polynomial conversion equation to obtain corresponding standard INR values. In some examples, the function parameters may be programmed into data storage device 146, e.g., into ROM 150, RAM 148, or HDD 152, permitting for system 100 to compute, record, and report INR values for any given test.

In some examples, processor 142 can perform the above first phase of the analysis to compute PT values in substantially real time, and can convert those PT values to INR values in negligible additional time. For example, PT and INR values can be reported in substantially no more time than is required for the test, e.g., no more than about two minutes after introduction of sample to slot.

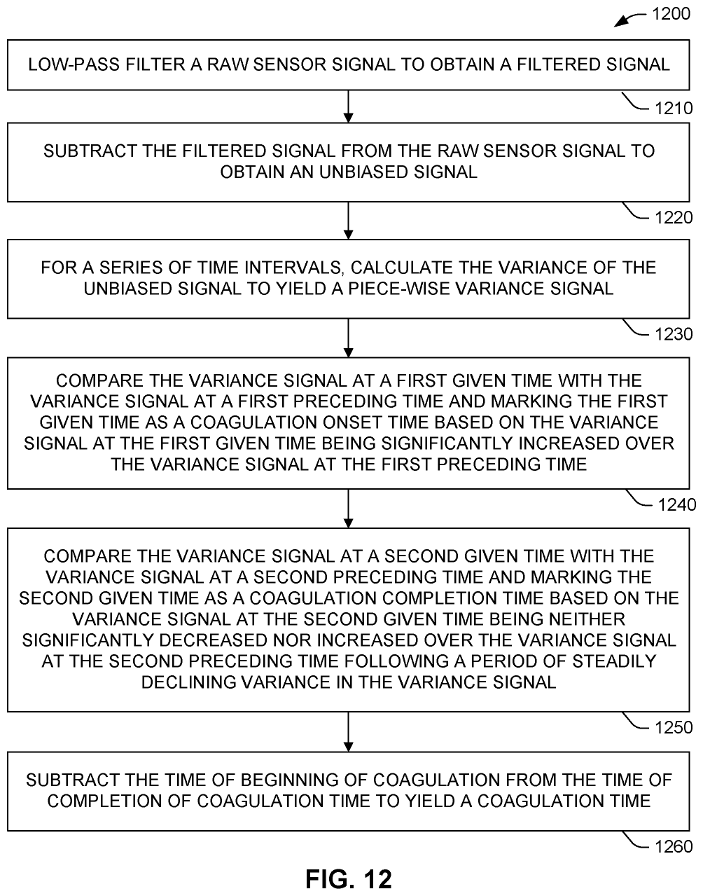

FIGS. 11 and 12 are flowcharts showing example methods of microfluidic coagulation testing. Examples of systems and methods are described herein with reference to flowchart illustrations and/or block diagrams of methods, apparatus (systems) and computer program products according to examples of the principles described herein. Some blocks of the flowchart illustrations and combinations of blocks in the flowchart illustrations may be implemented by computer-usable program code. The computer-usable program code may be provided to a processor of a general-purpose computer, special-purpose computer, or other programmable data-processing apparatus to produce a machine, such that the computer-usable program code, when executed via, for example, the processor 142 of the control/computation device 140 or other programmable data processing apparatus, implements and/or causes the functions or acts specified in the flowchart and/or block diagram block or blocks. In one example, the computer-usable program code may be embodied within a computer-readable storage medium, the computer-readable storage medium being part of the computer program product. In one example, the computer-readable storage medium is a non-transitory computer-readable medium.

The method 1100 of FIG. 11 may begin 1110 by introducing a fluid sample into a measurement device (e.g., device 120 of FIGS. 1 and 2) comprising at least one pinch point (e.g., pinch point 124) comprising a microfluidic channel (e.g., channel 128) of substantially consistent width (e.g., width W in FIG. 8) and height (e.g., height H in FIG. 3) connecting a slot (e.g., slot 122) and a chamber (e.g., chamber 134), the at least one pinch point permitting passage of sample from slot to chamber. Next, with a sensor in or near the at least one pinch point (e.g., sensor 132), transits of individual cells in the sample passing through the at least one pinch point can be measured 1120. Following measurement, a processor (e.g., processor 142) can be used to compute 1130 at least one metric indicative of a time period during which the flow of the sample transitions from substantially fluid flow to substantial cessation of flow (e.g., time period 1010 in FIG. 10).

The method 1200 of FIG. 12 provides one example of the computing 1130 in FIG. 11. Method 1200 can be performed can be performed, for example, by control/computation device 140 shown in FIG. 1, and specifically, using processor 142. Method 1200 can begin by low-pass filtering 1210 the raw sensor signal to obtain a filtered signal. Next, the filtered signal can be subtracted 1220 from the raw sensor signal to obtain an unbiased signal. Then, for a series of time intervals, the variance of the unbiased signal can be calculated 1230 to yield a piece-wise variance signal (e.g., of the form of variance signal 1002 or 1004 shown in FIG. 10).

Method 1200 can continue by comparing 1240 the variance signal at a first given time with the variance signal at a first preceding time and marking the first given time as a coagulation onset time based on the variance signal at the first given time being significantly increased over the variance signal at the first preceding time. Later, the variance signal at a second given time can be compared 1250 with the variance signal at a second preceding time and marking the second given time as a coagulation completion time based on the variance signal at the second given time being neither significantly decreased nor increased over the variance signal at the second preceding time following a period of steadily declining variance in the variance signal.

To yield a coagulation time, the time of beginning of coagulation can be subtracted 1260 from the time of completion of coagulation time. In some examples, this coagulation time can be, the at least one metric indicative of a time period during which the flow of the sample transitions from substantially fluid flow to substantial cessation of flow, as mentioned in FIG. 11. In other examples, the metric in FIG. 11 can be based at least in part on the coagulation time arrived at in 1260 of FIG. 12.

The method can additionally include adjusting at least one therapeutic regimen based on at least one reported metric generated as a result of the above-described test, e.g., adjusting an anticoagulant drug type, dosage or administration time based on a reported PT or INR value.

The methods described above thus include, as one example, a method that includes introducing a fluid sample into a measurement device including at least one pinch point including a microfluidic channel of substantially consistent width and height connecting a slot and a chamber, the at least one pinch point permitting passage of the fluid sample from the slot to the chamber. The method can further include measuring, with a sensor in or near the at least one pinch point, transits of individual cells in the sample passing through the at least one pinch point. The method can further include computing, at least in part with a processor and based on the measured transits, at least one metric indicative of a time period during which the flow of the sample transitions from substantially fluid flow to substantial cessation of flow.

Another example can include one or more non-transitory computer-readable media having instructions executable by a one or more processors, the instructions being programmed to perform a method including, for a series of time intervals, calculating the variance of a signal based on a sensor signal indicative of transits of individual cells in a sample passing through a microfluidic channel in a coagulation testing device, The performed method can further include comparing the variance at a first given time with the variance at a first preceding time and marking the first given time as a coagulation onset time based on the variance at the first given time being significantly increased over the variance at the first preceding time. The performed method can further include comparing the variance at a second given time with the variance at a second preceding time and marking the second given time as a coagulation completion time based on the variance signal at the second given time being neither significantly decreased nor increased over the variance at the second preceding time following a period of declining variance in the variance. The performed method can further include subtracting the time of beginning of coagulation from the time of completion of coagulation time to yield a coagulation time.

Because the systems, devices and methods described herein measure the flow of discrete sample features (e.g., red blood cells) directly, the method need not rely on secondary reactions (e.g., color change, the production of free electrons, etc.) to detect coagulation, thus eliminating the need for reagents (as in devices that use an amperogenic thrombin substrate to amplify an electric signal from coagulation), which reagents may be proprietary and/or expensive, which may require more quality control checks during test production, and which, moreover, could fail if not used in the proper conditions. The described systems, devices and methods, which work by measuring the presence and absence of red blood cell flow, also can be invariant to changes in hematocrit and other sample variability caused by differences in patient condition. For example, a change in the number of red blood cells present may change the frequency of peaks generated from cells flowing through the channel, but will not change the start and end time for cell flow to occur.

The method described herein may also provide a more sensitive test since it involves a direct measurement of cell flow rather than a secondary measurement of clotting such as thrombin production. In tests that look for successful conversion of a reagent to a product, only the production of thrombin is required, not complete coagulation. Such tests, therefore, do not require successful clotting to give a positive result, in contrast to the present method which measures clotting time by observing cessation of cell flow. In such reagent-requiring tests, the detection is farther removed from the coagulation process, and therefore in such tests the detection would be classified as secondary measurement, as opposed to the direct measurement employed in the method disclosed herein. The individual-cell sensitivity of the described systems, devices, and methods means that they can be made to use low quantities of activator (during fabrication) and sample (during testing). As an example, a single device 120 can be made using no more than five hundred nanoliters of tissue factor, for example, no more than two hundred nanoliters of tissue factor. As another example, a single test can require no more than five microliters of finger-prick whole blood. The present method further eliminates the need for frequent calibration, as may be required in mechanical clot detection used in benchtop tests.

Furthermore, the device architectures described herein are able to accommodate coagulation initializing tissue factor application. The particular architecture shapes and features described, particularly of the pinch point inlet 126, permit for tissue factor to be evenly coated so as not to clog pinch point or obstruct cell flow during the initial stages of a coagulation test, and to ensure even wetting upon introduction of sample into device 120.

In view of the foregoing, the microfluidic devices, systems, and methods disclosed herein provide effective coagulation testing solutions. The systems, devices and methods can provide automated determination of PT/INR values. The systems, devices, and methods can be adapted to be used with different sample types by adjusting the sizes and geometries of the features described herein and/or by using different coatings or active surfaces, providing versatility of use.

The preceding description has been presented to illustrate and describe examples of the principles described. This description is not intended to be exhaustive or to limit these principles to any precise form disclosed. Many modifications and variations are possible in light of the above teaching. What have been described above are examples. It is, of course, not possible to describe every conceivable combination of components or methods, but one of ordinary skill in the art will recognize that many further combinations and permutations are possible. Accordingly, the invention is intended to embrace all such alterations, modifications, and variations that fall within the scope of this application, including the appended claims. Additionally, where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, and the term “including” means including but not limited to. The term “based on” means based at least in part on.

What is claimed is:

1. A microfluidic coagulation testing die comprising:
a slot in a substrate permitting entry of a sample;
a sample collection chamber in the substrate;
at least one pinch point connecting the slot and the chamber, permitting passage of sample from slot to chamber, comprising:
a slot-side inlet,
a channel, having a length, of substantially consistent width and height, the width being between ten and fifteen micrometers and the height being between ten and twenty micrometers, and
a chamber-side outlet,
the pinch point having no angled corners in the inlet or channel in a plan view;
a sensor in or near the pinch point; and
coagulation-initializing activator coating at least a portion of an interior surface of any or all of the slot, the pinch point, and the chamber.

2. The die of claim 1, wherein the pinch point channel width is between ten and fifteen micrometers, the pinch point channel height is between ten and twenty micrometers, and the pinch point channel length is between five and fifteen micrometers.

3. The die of claim 1, having no more than about 200 nanoliters of activator.

4. The die of claim 1, wherein the substrate is silicon, and wherein the substrate is coupled to one or more layers of thin-film deposited SU-8 polymer.

5. The die of claim 1, having a chamber on only one side of the slot and not on the other side of the slot.

6. The die of claim 1, wherein the sensor comprises two electrodes on opposite sides of the at least one pinch point, the electrodes being arranged to measure disruption of an electric field between the electrodes by discrete features of sample passing through the at least one pinch point.

7. The die of claim 1, wherein the chamber comprises at least one nozzle to remove air and fluid from the chamber.

8. The die of claim 7, wherein the chamber comprises no more than two nozzles.

9. A device comprising:
a slot in a substrate, the slot having an interior surface area no greater than 1,200,000 square micrometers;
a chamber in the substrate, the chamber having an interior surface area no greater than 40,000 square micrometers;
at least one microfluidic pinch point connecting the slot and the chamber, the pinch point having an interior surface area no greater than 300 square micrometers, the pinch point comprising
an inlet from the slot,
a channel of substantially consistent width and height, the width being between ten and fifteen micrometers and the height being between ten and twenty micrometers, and
an outlet into the chamber, and
a sensor in or near the at least one pinch point to test sample fluid passing through the at least one pinch point.

10. The device of claim 9, wherein the interior surfaces of the slot, pinch point, and chamber are coated with an activator to initiate a coagulation process in the sample fluid.

11. The device of claim 9, wherein the chamber comprises at least one and no more than four nozzles to remove air and fluid from the chamber, and wherein no nozzle is located within one hundred micrometers of the pinch point outlet.

12. The device of claim 9, wherein the width of the channel is between ten and fifteen micrometers, the height of the channel is between ten and twenty micrometers, and the length of the channel is between five and fifteen micrometers.

13. A device comprising:
a slot, at least one pinch point, and at least one chamber in a substrate, the at least one pinch point comprising a microfluidic channel connecting the slot and the chamber;
wherein the interior surfaces of the slot, pinch point, and chamber are coated with a freeze-dried coagulation initiating tissue factor;
wherein the width of the channel is between ten and fifteen micrometers, the height of the channel is between ten and twenty micrometers, and the length of the channel is between five and fifteen micrometers; and
wherein the pinch point comprises at least one sensor to detect transits of individual red blood cells through the pinch point.

14. The device of claim 13, wherein the chamber comprises one or two nozzles to remove air and fluid from the chamber, and wherein no nozzle is located within one hundred micrometers of the pinch point outlet.

15. The device of claim 13, wherein the pinch point has an hourglass shape in a plan view.
